SEQUENCE LISTING (13 sequences)

SEQ ID NO: 1 - skipped

SEQ ID NO: 2 - DNA, 18 bp, synthetic construct
  source 1..18: mol_type = other DNA
attattattattattatt

SEQ ID NO: 3 - skipped

SEQ ID NO: 4 - DNA, 18 bp, synthetic construct
  source 1..18: mol_type = other DNA
aggaggaggaggaggagg

SEQ ID NO: 5 - DNA, 66 bp, synthetic construct
  source 1..66: mol_type = other DNA
aataataattgactgatttacggaagctgaataaggactgcttaggattgcgatgattcagctttc

SEQ ID NO: 6 - DNA, 75 bp, synthetic construct
  source 1..75: mol_type = other DNA
aataataataataataattgactgatttacggaagctgaataaggactgcttaggattgcgatgattcagctttc

SEQ ID NO: 7 - DNA, 66 bp, synthetic construct
  source 1..66: mol_type = other DNA
cctcctccttgactgatttacggaagctgaataaggactgcttaggattgcgatgattcagctttc

SEQ ID NO: 8 - DNA, 75 bp, synthetic construct
  source 1..75: mol_type = other DNA
cctcctcctcctcctccttgactgatttacggaagctgaataaggactgcttaggattgcgatgattcagctttc

SEQ ID NO: 9 - DNA, 43 bp, synthetic construct
  source 1..43: mol_type = other DNA
aataataataataataaggtggtggtggttgtggtggtggtgg

SEQ ID NO: 10 - DNA, 71 bp, synthetic construct
  source 1..71: mol_type = other DNA
cctcctcctcctcctcctcttccgatcttgagtagtcagggatcagtgcggttcgtgatggagatcggaag

SEQ ID NO: 11 - DNA, 57 bp, synthetic construct
  source 1..57: mol_type = other DNA
tgactgatttacggaagctgaataaggactgcttaggattgcgatgattcagctttc

SEQ ID NO: 12 - DNA, 26 bp, synthetic construct
  source 1..26: mol_type = other DNA
ggtggtggtggttgtggtggtggtgg

SEQ ID NO: 13 - DNA, 53 bp, synthetic construct
  source 1..53: mol_type = other DNA
cttccgatcttgagtagtcagggatcagtgcggttcgtgatggagatcggaag

(19) United States
(12) Patent Application Publication
(10) Pub. No.: US 2026/0166164 A1
(43) Pub. Date: Jun. 18, 2026

(54) NOVEL COMPOUND-PEPTIDE NUCLEIC ACID-APTAMER COMPLEX FOR TARGET PROTEIN DEGRADATION AND USE THEREOF

(71) Applicants: Korea University Research and Business Foundation, Seoul (KR); Korea Institute of Science and Technolgy, Seoul (KR)

(72) Inventors: Jun Seok LEE, Seoul (KR); Dong Hoon Kim, Seoul (KR); Yun Kyung Kim, Seoul (KR); Mohammad Faysal Al Mazid, Barishal (BD); Olha Viktorivna Shkel, Seoul (KR)

(73) Assignees: Korea University Research and Business Foundation, Seoul (KR); Korea Institute of Science and Technology, Seoul (KR)

(21) Appl. No.: 19/415,900

(22) Filed: Dec. 11, 2025

(30) Foreign Application Priority Data
Dec. 12, 2024 (KR) 10-2024-0184901

(51) Int. Cl. A61K 47/64; A61K 47/54; A61P 25/28

(52) U.S. Cl. CPC A61K 47/645; A61K 47/549; A61P 25/28

(57) ABSTRACT

Provided are a novel compound-peptide nucleic acid-aptamer complex for target protein degradation, and to a use thereof. The complex is prepared by binding a novel compound and a peptide nucleic acid to each other, and introducing an aptamer capable of complementarily binding to the peptide nucleic acid thereto. Thus, the complex targets and performs degradation of tau, nucleolin, and eIF4E as target proteins in cells, and thus may be valuably used in related industries.

### CROSS-REFERENCE TO RELATED APPLICATIONS

This application claims the priority of Korean Patent Application No. 10-2024-0184901 filed on Dec. 12, 2024, in the Korean Intellectual Property Office, the disclosure of which is incorporated herein by reference.

### INCORPORATION BY REFERENCE OF SEQUENCE LISTING

The instant application contains a Sequence Listing which has been filed electronically in xml format and is hereby incorporated by reference in its entirety. Said xml copy, created on Dec. 10, 2025, is named Q315585_sequence listing as filed and is 11,426 bytes bytes in size.

### BACKGROUND

### Field

The present disclosure relates to a novel compound-peptide nucleic acid-aptamer complex for target protein degradation and a use thereof.

### Description of the Related Art

Target Protein Degradation (TPD) is a next-generation treatment and is emerging as a promising strategy for proteins that it is difficult to develop into drugs. At present, more than 85% of the proteomes are not developed into drugs due to lack of a pocket structure thereof to which a ligand binds, and thus TPD which does not limit the range of target proteins, is attracting attention.

Proteolysis-targeting chimera (PROTAC)-based TPD strategy takes advantage of the proximate multifunctionality of ubiquitin E3 ligase and follows the proteasomal degradation process. Recently, it has been expanded to various TPD strategies utilizing molecular adhesive discovery and lysosomal degradation, such as lysosomal targeting chimeras (LYTAC), antibody-based PROTAC (AbTAC), covalently linked Nanobody-based PROTAC (GlueTAC), autophagy targeting chimeras (AUTAC and AUTOTAC), and the like. However, despite these various TPD platforms, major obstacles still exist in the target protein binding step. The current TPD strategy is to use i) known small molecule ligands, but without many proteins reported ligands, and ii) antibodies, but antibodies are applicable only to cell surface proteins, and iii) utilize intrinsic interaction partners based on molecular adhesion structures, but the development of novel substrates is a very challenging task.

Meanwhile, the aptamer has a unique advantage of systematically discovering a selective target binding aptamer through Systematic Evolution of Ligands by EXponential (SELEX), and the intracellular delivery of the oligonucleotide has been established by utilizing lipofectamine or liposomes. Nevertheless, aptamers have been limitedly utilized as functional units of PROTAC through PROTAC interaction with cancer cells, targeting of LYTAC proteins outside the cell or membrane, and chemical binding with VHL E3 ligase or cereblon. While conventional TPD techniques have shown the potential of aptamers, it is important that each degrader develops a novel material in which the binding between the aptamer and the E3 ligase binding ligand is individually performed.

Accordingly, the present inventors prepared a novel complex AptaGron in which an aptamer and a peptide nucleic acid bind to each other using a complementary base sequence linker, and performed fluorescence imaging of intracellular proteins and identified that using the novel complex AptaGron specifically degrades the protein.

### SUMMARY

A purpose of the present disclosure is to provide a compound represented by a following Chemical Formula 1.

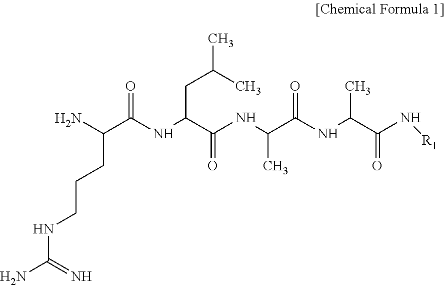

In the Chemical Formula 1,
R₁ is amino acid-R₂
R₂ is a peptide nucleic acid.

Another purpose of the present disclosure is to provide a complex for protein degradation, the complex comprising: the compound represented by the Chemical Formula 1; and an aptamer capable of complementarily binding to a peptide nucleic acid bound to the compound.

Still another purpose of the present disclosure is to provide a pharmaceutical composition for preventing or treating neurodegenerative diseases, the composition comprising the complex as an active ingredient.

The purposes of the present disclosure are not limited to the above-mentioned purposes, and other purposes and advantages of the present disclosure that are not mentioned may be understood based on the following descriptions, and will be more clearly understood based on the embodiments of the present disclosure. In addition, it will be readily appreciated that the purposes and advantages of the present disclosure may be realized by means indicated in the claims and combinations thereof.

The present disclosure provides a compound represented by the following Chemical Formula 1.

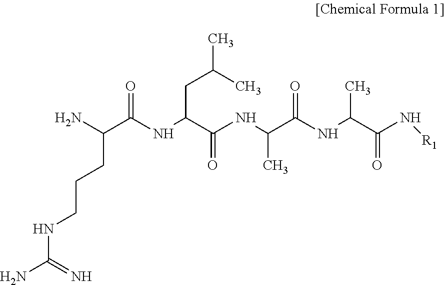

In the Chemical Formula 1,
R₁ is amino acid-R₂,
R₂ is a peptide nucleic acid.

In addition, the present disclosure provides a complex for protein degradation, the complex comprising: the compound represented by the Chemical Formula 1; and an aptamer capable of complementarily binding to a peptide nucleic acid bound to the compound.

In addition, the present disclosure provides a pharmaceutical composition for preventing or treating neurodegenerative diseases, the composition comprising the complex as an active ingredient.

The novel PROTAC-based complex of the present disclosure is prepared by binding the novel compound and the peptide nucleic acid to each other, and introducing the aptamer capable of complementarily binding to the peptide nucleic acid thereto. The novel PROTAC-based complex of the present disclosure has the effect of targeting and performing degradation of tau, nucleolin, and eIF4E as target proteins in cells, and thus can be usefully used in related industries.

The effects of the present disclosure are not limited to the aforementioned effects, and other effects, which are not mentioned above, will be apparently understood to a person having ordinary skill in the art from the following description.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 shows a structural formula of a novel complex AptaGron of the present disclosure;

FIG. 2 shows buffer stability of AptaGron of the present disclosure;

FIG. 3 shows the time stability of the AptaGron of the present disclosure;

FIG. 4 shows the degree of tau protein degradation of AptaGron of the present disclosure;

FIG. 5 shows tau-GFP degradation of AptaGron of the present disclosure;

FIG. 6 shows the tau protein degradation pattern in the TauBiFC mouse model brain sample of AptaGron of the present disclosure;

FIG. 7 shows intracellular fluorescence imaging on ^{GC18nt}AptaGron^{tau} of AptaGron of the present disclosure;

FIG. 8 shows the quantification value of the intracellular fluorescence signal on ^{GC18nt}AptaGron^{tau} of AptaGron of the present disclosure;

FIG. 9 shows the Tau-GFP degradation fluorescence signal on ^{GC18nt}AptaGron^{tau} of the AptaGron of the present disclosure;

FIG. 10 shows a fluorescence signal quantification value in the PAGE image of the AptaGron of the present disclosure;

FIG. 11 shows a Western blot on ^{AT18nt}AptaGron^{nuc} of AptaGron of the present disclosure;

FIG. 12 shows fluorescence imaging on ^{AT18nt}AptaGron^{nuc} of AptaGron of the present disclosure;

FIG. 13 shows a fluorescence signal quantification value on ^{AT18nt}AptaGron^{nuc} of AptaGron of the present disclosure;

FIG. 14 shows Western blots of eIF4E and tau with or without proteasome inhibitor MG132 cotreatment of AptaGron of the present disclosure;

FIG. 15 shows the fluorescence imaging on ^{GC18nt}AptaGron^{eIF4E} of AptaGron of the present disclosure; and

FIG. 16 shows a fluorescence signal quantification value on ^{GC18nt}AptaGron^{eIF4E} of the AptaGron of the present disclosure.

### DETAILED DESCRIPTION OF THE EMBODIMENT

Hereinafter, embodiments of the present disclosure will be described in detail with reference to the accompanying drawings. However, the following embodiments are presented as an example of the present disclosure, and when it is determined that a detailed description of a well-known and well-known technique or configuration to those skilled in the art may unnecessarily obscure the gist of the present disclosure, the detailed description may be omitted, and the present disclosure is not limited thereto. The present disclosure is capable of various modifications and applications within the description of the claims to be described later and the equivalent category interpreted therefrom.

In addition, terms (terminology) used in the present disclosure are terms used to appropriately express preferred embodiments of the present disclosure, and may vary depending on the intention of a user or an operator, or the practice of the field to which the present disclosure pertains. Therefore, the definitions of the terms should be made based on the contents throughout this specification. It will be further understood that the terms “comprise”, “comprising”, “include”, and “including” when used in this specification, specify the presence of the stated features, integers, operations, elements, and/or components, but do not preclude the presence or addition of one or more other features, integers, operations, elements, components, and/or portions thereof.

The present disclosure provides a compound represented by a following Chemical Formula 1.

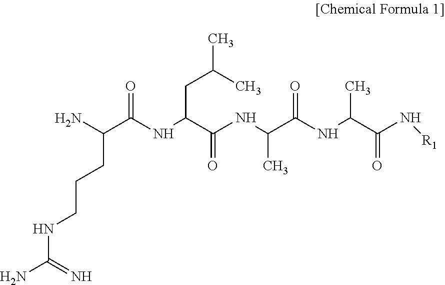

In the Chemical Formula 1,
R₁ is amino acid-R₂,
R₂ is a peptide nucleic acid.

The compound of the Chemical Formula 1 of the present disclosure may be synthesized using Arginine (R), Leucine (L), Alanine (A), Cysteine (C), or derivatives thereof.

According to an embodiment of the present disclosure, the amino acid may be selected from the group consisting of alanine, cysteine, or a combination thereof, and preferably may be alanine and cysteine binding thereto in the order of alanine and cysteine.

According to an embodiment of the present disclosure, the peptide nucleic acid may be a base sequence represented by a following Chemical Formula 2.

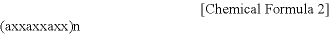

In the Chemical Formula 2,
a is Adenine,
x is Thymine (t) or Guanine (g),
n is 1 or 2.

According to an embodiment of the present disclosure, the peptide nucleic acid may be one selected from the base sequences represented by SEQ ID NOs: 1 to 4.

In the present disclosure, R₁ of the Chemical Formula 1 may be one selected from the following amino acid-base sequence group.

AC-attattatt,
AC-attattattattattatt,
AC-aggaggagg,
AC-aggaggaggaggaggagg.

In the amino acid-base sequence, A is alanine and C is cysteine.

In addition, the present disclosure provides a complex for protein degradation, the complex comprising: the compound represented by the Chemical Formula 1; and an aptamer capable of complementarily binding to a peptide nucleic acid bound to the compound.

The terms used in the present disclosure are defined as follows.

The “complex” of the present disclosure is a complex composed of two or more associated polypeptides. It is also called a multiprotein complex. The protein complex is distinct from the multienzyme complex in which the multicatalytic domain is found in a single polypeptide chain.

Hereinafter, the complex according to the present disclosure may be referred to as “AptaGron”.

The Peptide Nucleic Acid (PNA) of the present disclosure refers to a synthetic compound having a structure similar to that of DNA or RNA, and may recognize a gene sequence. PNA has a backbone composed of amino acids instead of the sugar-phosphate backbone of the typical DNA/RNA. Thus, PNA has higher stability and durability, and has a strong binding affinity to a specific base sequence.

The “complementary” of the present disclosure generally indicates a relationship in which two or more elements complement each other or act harmoniously. In the field of biology, in the formation of complementary base pairs of DNA, the complementary binding means A (adenine) binding to T (thymine) and means C (cytosine) binding to G (guanine). This principle plays an important role in DNA replication and transcription.

The “aptamer” of the present disclosure is a short DNA or RNA molecule that binds to a specific molecule, mainly a protein or a small compound. Aptamers are selectively found through SELEX (ligands derived from systemic evolution) and have high specificity and affinity. Aptamers are widely used in the fields of diagnosis, treatment, and research, and may play an important role, especially in drug delivery systems, or in recognizing and regulating target proteins in cancer treatment.

According to an embodiment of the present disclosure, the amino acid may be selected from the group consisting of alanine, cysteine, or a combination thereof.

According to an embodiment of the present disclosure, the amino acid may be a combination of alanine and cysteine in the order of alanine and cysteine.

According to an embodiment of the present disclosure, the base sequence may be a base sequence represented by the above Chemical Formula 2.

The peptide nucleic acid may be one selected from the base sequences represented by SEQ ID NOs: 1 to 4.

According to an embodiment of the present disclosure, the aptamer may be one selected from the base sequences represented by SEQ ID NOs: 5 to 10.

According to an embodiment of the present disclosure, the compound may be used to recruit intracellular proteins.

The “protein recruiting” of the present disclosure refers to a process in which a specific protein binds to another protein or molecule in a cell or gathers to a specific place. This process plays an important role in the regulation of cellular functions, signal transduction, and maintenance of cellular structure. The protein recruiting is regulated through protein-protein interactions, phosphorylation, or other chemical modifications.

According to an embodiment of the present disclosure, the aptamer may specifically target an intracellular protein.

According to an embodiment of the present disclosure, the protein may include tau (SEQ ID NO: 11), nucleolin (SEQ ID NO: 12), or eIF4E (eukaryotic initiation factor 4E; SEQ ID NO: 13).

The tau of the present disclosure is a protein mainly found in nerve cells and plays a role in maintaining the stability of microtubules. Tau protein plays an important role in neurodegenerative diseases such as Alzheimer's disease, and abnormal phosphorylation may form aggregates and interfere with cell function. Abnormal aggregates of these tau proteins are associated with neuronal apoptosis and cognitive decline.

The “nucleolin” of the present disclosure is a protein mainly present in the nucleus and plays an important role in cell growth, division, and survival. This protein is involved in the synthesis and processing of ribosomal RNA and also affects the regulation of gene expression in cells. Nucleolin tends to be overexpressed, especially in cancer cells, and is attracting attention as a target for chemotherapy. In addition, nucleolins are also involved in the stress response of cells, cell migration, and cell cycle regulation, which play an important role in various physiological and pathological processes.

The “eIF4E (gene translation initiation factor 4E, eukaryotic initiation factor 4E)” of the present disclosure is a protein that plays an important role in the early stages of protein translation. This protein binds to the 5′ cap structure of mRNA, contributing to translation initiation complex formation, and plays a key role in regulating protein synthesis. eIF4E plays an important role in cell growth and survival, and in particular, it is often overexpressed in cancer cells, so it may be involved in the development and progression of cancer. Therefore, eIF4E is being studied as a target for chemotherapy, and its inhibition is associated with growth inhibition of cancer cells. Functional regulation of eIF4E is also closely linked to signaling pathways, playing an important role in various physiological and pathological processes.

According to an embodiment of the present disclosure, the complex may degrade tau, nucleolin, or eIF4E (eukaryotic initiation factor 4E).

In addition, the present disclosure provides a pharmaceutical composition for preventing or treating neurodegenerative diseases, the composition comprising the complex as an active ingredient.

The term “prevention” used in the present disclosure refers to all acts of inhibiting symptoms of a specific disease or delaying the progression thereof by administering the composition of the present disclosure.

The term “treatment” used in the present disclosure refers to any act of improving or beneficially changing the symptoms of a specific disease by administration of the composition of the present disclosure.

The pharmaceutical composition of the present disclosure may further include an adjuvant in addition to the active ingredient. The adjuvant may be used without limitation as long as it is known in the art, but for example, a complete adjuvant of Freund or an incomplete adjuvant may be further included to increase the effect thereof.

The pharmaceutical composition according to the present disclosure may be prepared in a form in which an active ingredient is incorporated into a pharmaceutically acceptable carrier. Here, the pharmaceutically acceptable carrier includes a carrier, an excipient, and a diluent commonly used in the pharmaceutical field. Pharmaceutically acceptable carriers available in the pharmaceutical compositions of the present disclosure include, but are not limited to, lactose, dextrose, sucrose, sorbitol, mannitol, xylitol, erythritol, maltitol, starch, acacia rubber, alginate, gelatin, calcium phosphate, calcium silicate, cellulose, methyl cellulose, polyvinyl pyrrolidone, water, methylhydroxybenzoate, propylhydroxybenzoate, talc, magnesium stearate, and mineral oil.

The pharmaceutical composition of the present disclosure may be formulated into oral formulations such as powders, granules, tablets, capsules, suspensions, emulsions, syrups, aerosols, or the like, external preparations, suppositories, or sterile injectable solutions according to conventional methods.

In the case of formulation, it may be prepared using a diluent or excipient such as a commonly used filler, extender, binder, wetting agent, disintegrant, surfactant, etc. Solid formulations for oral administration include tablets, pills, powders, granules, capsules, and the like, and such solid formulations may be prepared by mixing the active ingredient with at least one excipient, for example, starch, calcium carbonate, sucrose, lactose, gelatin, and the like. In addition, lubricants such as magnesium stearate and talc may also be used in addition to simple excipients. Liquid formulations for oral administration include suspensions, solutions, emulsions, syrups, and the like, and various excipients, for example, wetting agents, sweeteners, air fresheners, preservatives, and the like, may be included in addition to water and liquid paraffin, which are generally used diluents. Formulations for parenteral administration include sterile aqueous solutions, non-aqueous solvents, suspensions, emulsions, lyophilized formulations, and suppositories. As the water-insoluble solvent and the suspending agent, propylene glycol, polyethylene glycol, vegetable oil such as olive oil, an injectable ester such as ethyl oleate, and the like may be used. Witepsol, tween 61, cacao butter, laurin butter, glycerogelatin, and the like may be used as the base of the suppository.

The pharmaceutical composition according to the present disclosure may be administered to a subject by various routes. Any mode of administration may be expected, for example, by oral, intravenous, muscle, subcutaneous, or intraperitoneal injection.

The dosage of the pharmaceutical composition according to the present disclosure is selected in consideration of the age, weight, sex, physical condition, etc. of the subject. It is obvious that the concentration of the active ingredient contained in the pharmaceutical composition may be variously selected according to the target, and preferably, the active ingredient is contained in a concentration of 0.01 to 5,000 μg/ml in the pharmaceutical composition. When the concentration of the active ingredient is smaller than 0.01 μg/ml, the pharmaceutical activity may not appear, and when the concentration of the active ingredient exceeds 5,000 μg/ml, it may be toxic to the human body.

According to an embodiment of the present disclosure, the neurodegenerative disease may be any one selected from the group consisting of Alzheimer's disease, Parkinson's disease, Creutzfeldt-Jakob disease (CJD), Hallervorden-Spatz disease, Huntington's disease, multi-system atrophy, dementia, Frontotemporal dementia, amyotrophic lateral sclerosis, spinal muscular atrophy, Spinocerebellar Ataxia (SCA), meningoencephalitis, bacterial meningoencephalitis, viral meningoencephalitis, CNS autoimmune disorder, multiple sclerosis (MS) and acute ischemic injury.

Hereinafter, the present disclosure will be described in more detail by the following examples. However, these examples are only for illustrating the present disclosure, and the scope of the present disclosure is not limited by these examples.

### <Example 1> Preparation of Novel Complex AptaGron

A novel complex in which the compound represented by the following Chemical Formula 1 and the aptamer were combined with each other was prepared.

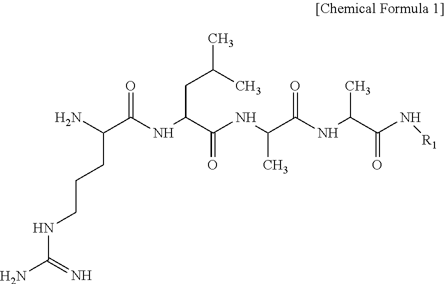

In the Chemical Formula 1,
R₁ is amino acid-R₂,
R₂ is a peptide nucleic acid.

Specifically, a substituent in which the amino acid and the peptide nucleic acid (PNA) sequence were sequentially bound to each other was introduced to a R₁ position of the compound. In this regard, the amino acid was a combination of Alanine (A) and Cysteine (C) in this order. The peptide nucleic acid employed the SEQ ID NOs: 1 to 4 of Table 1 below, and was complementarily bound to the 5′-end of the aptamer to form one complex in which the aptamer and the compound are connected to each other.

The base sequences represented by SEQ ID NOs: 1 to 4 of the peptide nucleic acid of the present disclosure are shown in Table 1 below.

TABLE 1

| No. | PNA sequence | |
|---|---|---|
| SEQ ID | x = T, | att att att |
| NO: 1 | n = 1 | (HPLC 99.9%, MS found (M + 1): |
| | | 3026.2) |
| SEQ ID | x = T, | att att att att att att |
| NO: 2 | n = 2 | (HPLC 99.9%, MS found (M + 1): |
| | | 5450.2) |
| SEQ ID | X = G, | agg agg agg |
| NO: 3 | n = 1 | (HPLC 99.9%, MS found (M + 1): |
| | | 3177.0) |
| SEQ ID | x = G, | agg agg agg agg agg agg |
| NO: 4 | n = 2 | (HPLC 99.9%, MS found (M + 1): |
| | | 5749.8) |

In addition, the base sequences represented by SEQ ID NOs: 5 to 10 of the aptamer of the present disclosure are shown in Table 2 below.

TABLE 2

| No. | Name | Aptamer sequence | |
|---|---|---|---|
| SEQ ID | ^{AT9nt}aptamer^{tau} | x = t, | 5′ - aat aat aat tga ctg att tac gga agc |
| NO: 5 | | n = 1 | tga ata agg act gct tag gat tgc gat gat |
| | | | tca gct ttc -3′ |
| | | | (Cal mw: 20444.3, Detected mw: 20445.00) |
| SEQ ID | ^{AT18nt}aptamer^{tau} | x = t, | 5′ - aat aat aat aat aat aat tga ctg att |
| NO: 6 | | n = 2 | tac gga agc tga ata agg act gct tag gat |
| | | | tgc gat gat tca got ttc -3′ |
| | | | (Cal mw: 23236.2, Detected mw: 23237.90) |
| SEQ ID | ^{GC9nt}aptamer^{tau} | x = g, | 5′ - cct cct cct tga ctg att tac gga agc |
| NO: 7 | | n = 1 | tga ata agg act gct tag gat tgc gat gat |
| | | | tca gct ttc -3′ |
| | | | (Cal mw: 20300.2, Detected mw: 20301.10) |
| SEQ ID | ^{GC18nt}aptamer^{tau} | x = g, | 5′- cct cct cct cct cct cct tga ctg att |
| NO: 8 | | n = 2 | tac gga agc tga ata agg act gct tag gat |
| | | | tgc gat gat tca got ttc -3′ |
| | | | (Cal mw: 22947.9, Detected mw: 22948.60) |
| SEQ ID | ^{AT18n}taptamer^{nuc} | x = t, | 5′ - aat aat aat aat aat aag gtg gtg gtg |
| NO: 9 | | n = 2 | gtt gtg gtg gtg gtg g -3′ |
| | | | (Cal mw: 13551.8, Detected mw: 13551.70) |
| SEQ ID | | x = g, | 5′ - cct cct cct cct cct cct ctt ccg atc |
| NO: 10 | ^{GC18nt}aptamer^{eIF} | n = 2 | ttg agt agt cag gga tca gtg cgg ttc gtg |
| | | | atg gag atc gga ag -3′ |
| | | | (Cal mw: 21795.1, Detected mw: 21793.50) |

Specifically, the compound to which amino acid-PNA was bound at a concentration of 5 M and the aptamer at a concentration of 5 μM were mixed with each other in PBS buffer (pH 7.4), the mixture was heated and denatured at 95° C. for 5 minutes, cooled to 30° C. at a rate of 1.38° C. per minute, and then the mixture was incubated at 30° C. for 10 minutes and maintained at 10° C.

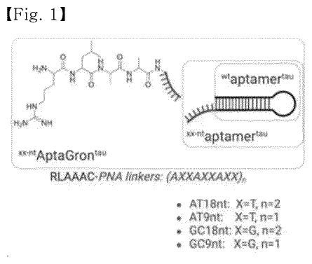

The structure of the novel complex AptaGron of the present disclosure is shown in FIG. 1.

### <Example 2> Cell Stability Analysis

The HEK293T and MCF7 cells of the present disclosure were added to Dulbecco's modified Eagle medium (DMEM) containing 10% fetal bovine serum (FBS) and penicillin-streptomycin, followed by incubation at 37° C. under 5% concentration of CO₂ gas.

Specifically, MCF7 and HEK293T cells were dispensed in a 96-well plate (Corning) at a density of 4×10⁴ cells per well, incubated at 37° C. for 24 hours, and then the cells were washed twice with cold Dulbecco's Phosphate-Buffered Saline (DPBS). After lysis buffer containing 50 mM Tris⋅HCl pH 7.4, 150 mM NaCl, 1% TritonX, 1 mM EDTA, 1 mM DTT, and 1×protease inhibitor cocktail was added to the cells in an ice bath, the cell lysate was centrifuged at 13,000 rpm at 4° C. for 15 min and the protein supernatant was collected. The cell lysate was mixed with 4×SDS loading buffer and heated at 95° C. for 5 min. 20 μL of the heated lysate was loaded onto a SDS-PAGE gel and treated on a PVDF membrane. The membrane was blocked with 3% BSA in TBST (Tris-Buffered Saline with 0.01% Tween-20), incubated with the primary antibody at 4° C. for 24 h, and then the membrane was treated with a phosphor-conjugated secondary antibody for 1 h at room temperature and then visualized using a ChemiDoc MP imaging system.

To identify the aptamer stability in buffer or cell culture, a 10 μM concentration of each PNA and aptamer mixture was diluted in PBS buffer (pH 7.4) and incubated at 37° C. for 48 hours. The reaction mixture was analyzed with a 4% agarose gel.

In addition, a 10 μM concentration of each PNA and aptamer mixture was incubated at 37° C. for 1 hour, 2 hours, 4 hours, 6 hours, 10 hours, 20 hours and 24 hours to obtain a sample. Samples were processed using 3′-ACGCTACTAAGTCGAAAG-5′ (reverse primer) and 5′-GACTGATTTACGGAAGCT-3′ (forward primer). The resulting DNA was analyzed with a 4% agarose gel.

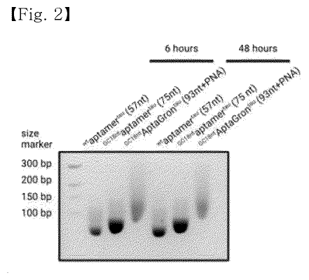

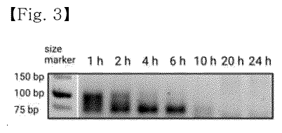

As a result, as shown in FIGS. 2 and 3, it was identified that all of ^{wt}aptaGron^{tau}, ^{GC18nt}aptaGron^{tau}, and hybridized ^{GC18nt}AptaGron^{tau} were not degraded during the 48-hour incubation period, and AptaGron or aptamer bands could be detected in HEK293T cells for up to 6 hours.

### <Example 3> Inhibition of Target Protein

In order to monitor the degradation of the target protein, GFP-fused tau (Tau-GFP) capable of overexpression in HEK293T cells was used.

Specifically, the cultured HEK293T cells were transfected with the Tau-GFP expression construct and then grown in Dulbecco of modified Eagle medium (DMEM) to a density of 70 to 80%. Cells were harvested and centrifuged at 14,000 rpm for 20 min to obtain supernatant containing overexpressed tau protein, and then 100 μM ^{GC18nt}AptaGron^{tau} or 10 μM MG132 protein degradation inhibitor (Sigma, M7449) was added and incubated at 37° C. for 16 h to obtain soluble protein lysate. After incubation, the lysate samples were subjected to SDS-PAGE and subjected to Western blot analysis. Degradation mediated by AptaGron was monitored by the GFP signal in fluorescent PAGE images.

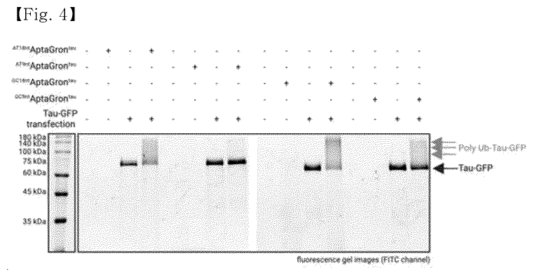

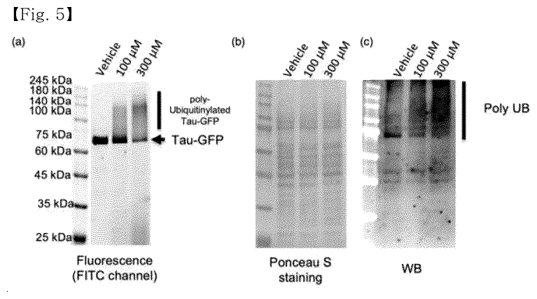

As a result, as shown in FIGS. 4 and 5, it was identified that Tau-GFP decreased due to protein degradation in the aptamer of the 18-nt length linker compared to the 9-nt length linker, and in the case of ^{AT9nt}AptaGron^{tau}, tau protein degradation hardly occurred due to the short length and low affinity due to the smaller hydrogen bond pair.

### <Example 4> Protein Targets Related to Neurodegenerative Diseases

### 4-1. Tau Protein Degradation

Three pathogen-free TauP301L-BiFC-transformed 12-month-old mouse models were reared at 12-h intervals day and night, perfused with 0.9% saline, and then brains were extracted. After the brains were weighed, they were suspended in RIPA lysis buffer with a mixture of protein degradation enzymes and phosphohydrolase inhibitors. Brain tissue was ground using a 2 mL glass dunk homogenizer and incubated at 4° C. for 2 hours. The homogenized mixture was centrifuged at 20,000 g for 20 min at 4° C., and the supernatant was collected and stored at 80° C. For immunoblot analysis, 25 μg and 100 μM ^{GC18nt}AptaGron^{tau} of each lysate were mixed and incubated at 37° C. for 24 h to obtain brain protein lysate. All experiments were performed in three replicates, and the data were analyzed using Student's t-test. If the p-value was less than 0.05, it was considered statistically significant.

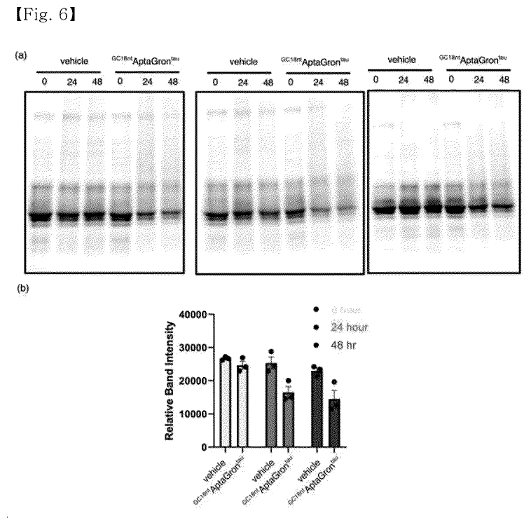

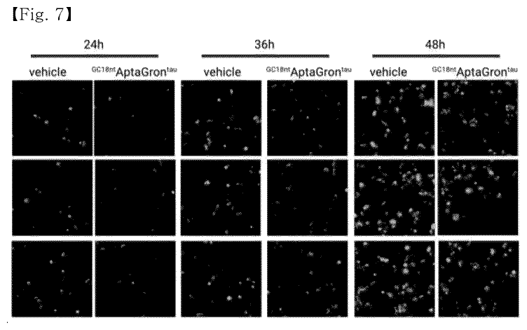

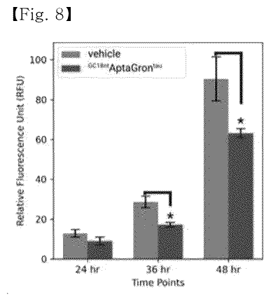

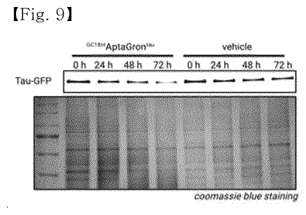

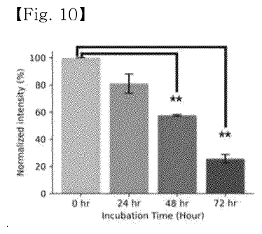

As a result, as shown in FIG. 6 to FIG. 10, it was identified that the cells treated with ^{GC18nt}AptaGron^{tau} degraded the tau protein after 24 hours, and showed a lower fluorescence intensity of the Tau-GFP protein compared to the control group in both the biocellular fluorescence image and the PAGE analysis.

### 4-2. Nucleolin Degradation

A 10 μM aptamer was prepared by binding a 18-nt linker to tau (^{wt}aptamer^{tau}: 57-nt), nucleolin (^{wt}aptamer^{nuc}: 26-nt), and elF4E (^{wt}aptamer^{elF}: 53-nt) as target proteins for binding to the aptamer. The information on the target protein of the present disclosure is shown in Table 3 below.

TABLE 3

| | Target | Intracellular | | | |
|---|---|---|---|---|---|
| No. | protein | localization | Name | KD (nM) | sequence (5′-3′) |
| SEQ ID | tau | Cytoplasm | IT2a | 19.5 ± 1.7 | TGACTGATTTACGGAAGC |
| NO: 11 | | | | | TGAATAAGGACTGCTTAG |
| | | | | | GATTGCGATGATTCAGCT |
| | | | | | TTC |
| SEQ ID | nulceolin | Nucleus | AS1411 | not | GGTGGTGGTGGTTGTGGT |
| NO: 12 | | | | determined | GGTGGTGG |
| SEQ ID | eIF4E | Cytoplasm | eIF4e | not | CTTCCGATCTTGAGTAGT |
| NO: 13 | | | Apt 1 | determined | CAGGGATCAGTGCGGTTC |
| | | | | | GTGATGGAGATCGGAAG |

Specifically, PNA and ^{AT18nt}aptamer^{nuc} were mixed with each other and then the mixture was thermally annealed to obtain 10 μM ^{AT18nt}AptaGron^{nuc}, which was mixed with the total cell lysate of MCF7 breast cancer cells and subsequently cultured. The degradation pattern of nucleolin was monitored by using Western blot analysis.

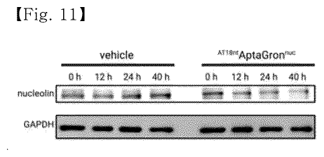

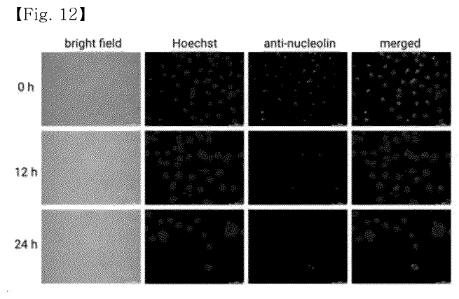

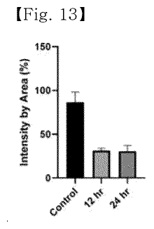

As a result, as shown in FIGS. 11 to 13, nucleolin was found in the intracellular nucleus, and it was identified that the expression of nucleolin decreased as the incubation time increased, and thus the fluorescence decreased.

4-3. eIF4E Degradation

Using the same protocol in Table 3 above, the degradation pattern of the eIF4E of AptaGron for the eIF4E configured with the 18-nt link was monitored.

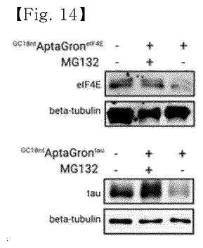

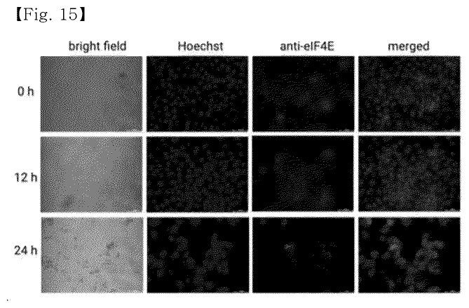

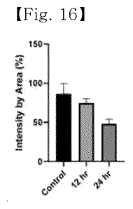

As a result, as shown in FIGS. 14 to 16, it was identified that ^{GC18nt}AptaGron^{eIF4E} degraded eIF4E in cells, thereby reducing fluorescence, and eIF4E degradation was inhibited when a MG132 protein degradation inhibitor was added.

Therefore, the PROTAC-based novel complex of the present disclosure was prepared by binding the novel compound and the peptide nucleic acid to each other, and introducing the aptamer capable of complementarily binding to the peptide nucleic acid thereto. Thus, the effect thereof targeting and degrading tau, nucleolin, and eIF4E, which are target proteins in cells thereof was identified.

As described above, although specific embodiments of the present disclosure have been described in detail, those skilled in the art who understand the spirit of the present disclosure may easily propose other reciprocal disclosures or other embodiments included in the spirit of the present disclosure by adding, changing, deleting, or the like other components within the same spirit. Therefore, it should be understood that the embodiments described above are exemplary in all aspects and are not limited thereto. The scope of the present disclosure is indicated by the scope of the patent claims to be described later rather than the detailed description, and it should be interpreted that all changes or modifications derived from the meaning and scope of the scope of the patent claims and the concept of equality thereof are included in the scope of the present disclosure.

What is claimed is:

1. A compound represented by a following Chemical Formula 1: 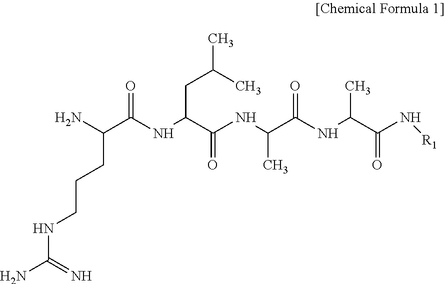
where in the Chemical Formula 1, R₁ is amino acid-R₂,
R₂ is a peptide nucleic acid.

2. The compound of claim 1, wherein the amino acid is selected from the group consisting of alanine, cysteine, and a combination thereof.

3. The compound of claim 2, wherein the amino acid is a combination of alanine and cysteine in the order of alanine and cysteine.

4. The compound of claim 1, wherein the peptide nucleic acid is a base sequence represented by a following Chemical Formula 2: 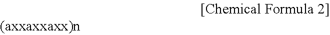
where in the Chemical Formula 2,
a is Adenine,
x is Thymine (t) or Guanine (g),
n is 1 or 2.

5. The compound of claim 4, wherein the peptide nucleic acid is one selected from the base sequences represented by SEQ ID NOs: 1 to 4.

6. A complex for protein degradation, the complex comprising:
a compound represented by a following Chemical Formula 1; and
an aptamer capable of complementarily binding to a peptide nucleic acid bound to the compound: 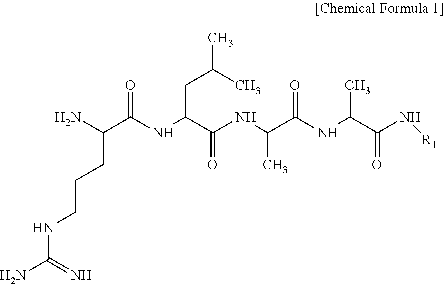
where in the Chemical Formula 1, R₁ is amino acid-R₂,
R₂ is the peptide nucleic acid.

7. The complex of claim 6, wherein the amino acid is selected from the group consisting of alanine, cysteine, or a combination thereof.

8. The complex of claim 7, wherein the amino acid is a combination of alanine and cysteine in the order of alanine and cysteine.

9. The complex of claim 6, wherein the peptide nucleic acid is a base sequence represented by a following Chemical Formula 2: 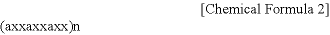
where in the Chemical Formula 2,
a is Adenine,
x is Thymine (t) or Guanine (g),
n is 1 or 2.

10. The complex of claim 9, wherein the peptide nucleic acid is one selected from the base sequences represented by SEQ ID NOs: 1 to 4.

11. The complex of claim 6, wherein the aptamer is one selected from the base sequences represented by SEQ ID NOs: 5 to 10.

12. The complex of claim 6, wherein the compound recruits an intracellular protein.

13. The complex of claim 6, wherein the aptamer specifically targets an intracellular protein.

14. The complex of claim 13, wherein the protein includes tau, nucleolin or eIF4E (eukaryotic initiation factor 4E).

15. The complex of claim 6, wherein the complex degrades tau, nucleolin or eIF4E (eukaryotic initiation factor 4E).

16. A pharmaceutical composition for preventing or treating a neurodegenerative disease, the pharmaceutical composition comprising the complex of claim 6 as an active ingredient.

17. The pharmaceutical composition of claim 16, wherein the neurodegenerative disease is one selected from the group consisting of Alzheimer's disease, Parkinson's disease, Creutzfeldt-Jakob disease (CJD), Hallervorden-Spatz disease, Huntington's disease, multi-system atrophy, dementia, Frontotemporal dementia, amyotrophic lateral sclerosis, spinal muscular atrophy, Spinocerebellar Ataxia (SCA), meningoencephalitis, bacterial meningoencephalitis, viral meningoencephalitis, CNS autoimmune disorders, multiple sclerosis (MS), and acute ischemic injury.

18. A method for preventing or treating a neurodegenerative disease, the method comprising administering to a subject in need thereof a pharmaceutical composition comprising the complex of claim 6 as an active ingredient.
